(19) United States
(12) Patent Application Publication
(10) Pub. No.: US 2025/0161294 A1
(43) Pub. Date: May 22, 2025

(54) COMPOSITIONS AND METHODS FOR TREATMENT OF OCULAR DISORDERS

(71) Applicant: Bausch + Lomb Ireland Limited, Dublin (IE)

(72) Inventors: Martin J. Coffey, Pittsford, NY (US); Vijay K. Singh, Webster, NY (US); Brian Rohrs, Fairport, NY (US)

(21) Appl. No.: 18/949,319

(22) Filed: Nov. 15, 2024

Related U.S. Application Data
(60) Provisional application No. 63/600,337, filed on Nov. 17, 2023.

(51) Int. Cl. A61K 31/4725; A61K 9/00; A61K 47/06

(52) U.S. Cl. CPC A61K 31/4725; A61K 9/0048; A61K 47/06

(57) ABSTRACT

Disclosed herein are ophthalmic compositions, such as non-aqueous liquid ophthalmic compositions, comprising lifitegrast, and at least one semifluorinated alkane, such as 1-perfluorohexyloctane (F6H8), as well as methods of treatment or prevention of at least one ocular disorder, such as dry eye disease, with such compositions. The ophthalmic compositions may further be provided in a container closure system, such as a polypropylene (PP) bottle, or in a kit.

### CROSS-REFERENCE TO RELATED APPLICATIONS

This application claims the benefit of U.S. Patent Application 63/600,337, filed Nov. 17, 2023, the complete contents of which are incorporated herein by reference.

### BACKGROUND

Keratoconjunctivitis sicca, also known as dry eye disease, is a multifunctional disorder of the tear film, and ocular surface which results in discomfort, visual disturbance, and often even in ocular surface damage. The two mechanisms that are being discussed as pivotal in the etiology of this disease and which also appear to reinforce each other mutually are tear hyperosmolarity and tear film instability. Hyperosmolar tear fluid can result from excessive tear film evaporation or reduced aqueous flow. It activates an inflammatory cascade and causes the release of inflammatory mediators into the tear fluid, with multiple pathophysiological effects eventually leading to further increased tear film evaporation and tear film instability. Thus, tear film instability can be a consequence of hyperosmolarity. Alternatively, tear film instability can also develop through its own etiological pathway, for example via abnormalities of the lipid layer composition, such as from meibomian gland disease.

The inflammation cycle is one of the key processes that maintain and potentially progress the dry eye disease. Depending on the severity of the condition, patients often develop a reversible squamous metaphase and punctate erosions of the ocular epithelium. Secondary diseases whose development may be triggered by dry eye disease include filamentary keratitis, microbial keratitis, corneal neovascularisation, and ocular surface keratinisation.

Two major categories of dry eye disease are distinguished today, which are aqueous-deficient DED and evaporative DED. These conditions are not necessarily mutually exclusive.

Within the class of aqueous-deficient forms of dry-eye disease, two major subtypes are differentiated, Sjögren and non-Sjögren. Sjögren syndrome patients suffer from autoimmune disorders in which the lacrimal glands are invaded by activated T-cells, which leads not only to keratoconjunctivitis sicca but also to a dry mouth condition. The Sjögren syndrome can be a primary disease or result from other autoimmune diseases such as systemic lupus erythrematosus or rheumatoid arthritis. Non-Sjögren patients suffering from an aqueous-deficient DED usually have a lacrimal gland insufficiency, lacrimal duct obstruction or reflex hyposecretion.

The second major class, evaporative DED, is also somewhat heterogeneous and can develop as a result of diverse root causes. Causes associated with increased evaporative loss of the tear film include meibomian gland disease, eyelid aperture disorders, blink disorders (as in Parkinson disease) or ocular surface disorders (as in allergic conjunctivitis). In particular, meibomian gland diseases are prevalently associated with evaporative dry eye disease. For example, meibomian gland dysfunction can result in changes in the quantitative or qualitative secretion of the lipid components required for the tear film. This in turn can lead to a failure in forming a stable and continuous tear film, which is followed by evaporative loss and hyperosmolarity. Meibomian gland dysfunction can often be characterized by gland obstruction and clogging through hyperkeratinisation of the gland and increased viscosity of the meibum. Dysfunction can arise from a primary lid-margin related disease or a secondary disease arising from systemic disorders such as acne rosacea or seborrheic dermatitis.

Typically, ophthalmic compositions for treatment of dry eye disease are administered as eye drops, with one or more drops of the composition being applied to an eye of the subject suffering from or susceptible to ocular disorders one or more times per day, although the frequency of administration of such compositions may be dependent on multiple factors, including the makeup of the particular composition.

Of particular importance for efficacy and commercialization of ophthalmic compositions is stability. Maintenance of efficacy and stability of ophthalmic compositions may be required to meet various federal health and safety regulations, e.g., potency, impurities, shelf life testing, sterility, etc. For example, ophthalmic compositions may be required to contain expiration dates posted on their container, which may be predicated on the stability of the active ingredients and other conditions inherent in the formulation and environmental exposures of the product.

Of further importance, drug solutions in eye drop bottles can get contaminated during use due to contact of the tip with hands or tears while instilling the drops. The high potential for the contamination after opening the bottles has led to regulations that require the addition of an antimicrobial agent in multi-dose eye drop formulations. Several preservatives have been researched and used in commercial formulations, including: alcohols, parabens, EDTA, chlorhexidine, and quaternary ammonium compounds. In addition to antimicrobial efficacy, the preservatives require suitable physical properties for incorporation into the formulations, such as chemical and thermal stability, compatibility with the eye drop container and other compounds in the formulation, and, more importantly, negligible toxicity to ocular tissues.

The potential for ocular damage from the preservatives is particularly high among patients suffering from chronic diseases that require daily eye drop instillations for periods of years to decades, such as dry eye disease. Indeed, most preservatives are problematic for dry eye disease patients in that they have a potential to negatively affect the ocular surface, thus counteracting the therapeutic intent. This is particularly relevant for patients with moderate to severe dry eye disease symptoms who may require frequent use for symptom relief, as well as patients who require multiple preserved topical medicaments.

As an alternative, single-dose containers for the administration of non-preserved formulations were developed. These are however less cost-efficient and convenient to handle for the patient than the conventional multi-dose bottle. The formulations of the present invention may be packaged as either a single dose product or a multi-dose product. The single dose product is sterile prior to opening of the package and all the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications, and incorporated herein by reference. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye disease. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

Lifitegrast (the active ingredient of Xiidra®) is a novel small molecule lymphocyte function-associated antigen 1 (LFA-1) antagonist that has recently been approved by the US Food and Drug Administration for the treatment of signs and symptoms of dry eye disease. It has the following structure:

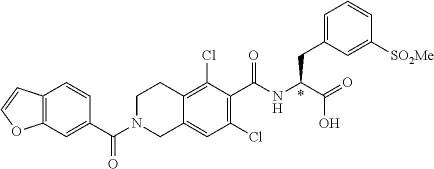 [SMILES: CS(=O)(=O)c1cccc(C[C@H](NC(=O)c2c(Cl)cc3c(c2Cl)CCN(C(=O)c2ccc4ccoc4c2)C3)C(=O)O)c1]

The current understanding of the mechanism of action of lifitegrast is that it decreases T cell-mediated inflammation associated with dry eye disease by blocking the interaction between the integrin LFA-1 and intercellular adhesion molecule 1 (ICAM-1), thereby preventing inflammatory cell activation and migration. Xiidra is currently provided as a non-preserved 5.0% ophthalmic solution in single-dose units and administered to each eye twice daily (b.i.d.; ˜ 12 hours apart).

As with all pharmaceuticals, lifitegrast should be provided in a form that is chemically pure, stable, and in a physical form suitable for administration into a subject. Various solutions to enhance the stability of lifitegrast have included the inclusion of various buffers and/or anti-oxidants (e.g., a thiosulfate salt) or sparging the formulation with an inert gas.

Despite the success of Xiidra in meeting the needs of many patients suffering from dry eye disease, there remains a need for improved dry eye disease treatments, particularly those that are stable and having lesser concentrations of or even substantially free of potentially irritating anti-oxidants and/or preservatives.

### SUMMARY

Disclosed herein are ophthalmic compositions, such as non-aqueous liquid ophthalmic compositions, comprising at least one small molecule lymphocyte function-associated antigen 1 (LFA-1) antagonist, such as lifitegrast, and at least one semifluorinated alkane, such as 1-perfluorohexyloctane (F6H8). The ophthalmic compositions may be used to treat and/or temporarily prevent an ocular condition, such as dry eye disease, in a single-dose or multi-dose unit composition. The ophthalmic compositions are stable for each active ingredient, provide efficient drug delivery, and are well tolerated by the eye. The ophthalmic compositions may be provided in a sealed single unit dose or in a multi-dose container closure system, such as a polypropylene (PP) bottle.

### BRIEF DESCRIPTION OF DRAWINGS

FIG. 1 shows cumulative lifitegrast concentrations (μg/g) in EpiCorneal™ tissue after 4 hours.

FIG. 2 shows the concentration (ng/g) of lifitegrast measured in cornea over time (0-8 hours).

### DISCLOSURE

The present inventors have surprisingly discovered that improved, stable ophthalmic compositions may be provided comprising (a) lifitegrast; and (b) at least one semifluorinated alkane. These compositions are preferably preservative-free and/or free of anti-oxidants. Without wishing to be bound by theory, it is believed that, not only does the at least one semifluorinated alkane provide its own ocular disorder treatment and/or prevention effect (thus intersecting the disease cycle with effects on both inflammation and tear evaporation), but it also acts as a stable, non-aqueous vehicle or carrier for the lifitegrast. The non-aqueous nature of some embodiments of the present invention also allows for the reduction or elimination of the need for additional ingredients, such as anti-oxidants and/or preservatives. It is thus the unique combination of the lifitegrast and the at least one semifluorinated alkane that provides the improved, stable ophthalmic compositions described herein. Furthermore, the compositions can achieve similar ocular exposure as aqueous lifitegrast compositions but with only one third of the dose. In some embodiments, the ophthalmic compositions may be non-aqueous liquid compositions comprising (a) lifitegrast, and (b) at least one semifluorinated alkane, such as 1-perfluorohexyloctane (F6H8).

Further disclosed herein are methods for the treatment and/or temporary prevention of dry eye disease, comprising administering to a patient in need thereof an effective amount of an ophthalmic composition comprising (a) lifitegrast, and (b) at least one semifluorinated alkane, such as 1-perfluorohexyloctane (F6H8).

Similarly, provided are: ophthalmic compositions comprising (a) lifitegrast and (b) at least one semifluorinated alkane, for use in a method for the treatment or temporary prevention of dry eye disease; and the use of (a) lifitegrast and (b) at least one semifluorinated alkane in the manufacture of an ophthalmic composition for the treatment or temporary prevention of dry eye disease.

Further disclosed herein are methods for preparing an ophthalmic composition, comprising steps of: (a) providing a first composition comprising lifitegrast and at least one semifluorinated alkane, such as 1-perfluorohexyloctane (F6H8), wherein the lifitegrast has settled, at least in part, within the first composition; and (b) agitating the first composition to give a suspension of lifitegrast, thereby providing the ophthalmic composition. This process will typically be performed in a container closure system such as a bottle, and the agitation will be shaking (or serial inversions) by hand to achieve redispersal of a suspension which has settled, at least in part, from which droplets may then be dispensed.

Further disclosed herein are methods for preparing an ophthalmic composition, comprising a step of mixing lifitegrast with at least one semifluorinated alkane, such as perfluorohexyloctane. Also disclosed are methods for packaging an ophthalmic composition, comprising steps of: (a) providing a suspension of lifitegrast in at least one semifluorinated alkane, such as perfluorohexyloctane; (b) placing some or all of the suspension into an open container; and (c) closing the container. Step (a) may involve mixing lifitegrast with the at least one semifluorinated alkane to form the suspension.

Further disclosed is the use of a SFA, such as F6H8, as a preservative for lifitegrast in an ophthalmic composition (and, in particular, in a multi-dose ophthalmic composition e.g. located within a container closure system, such as a bottle).

These and other features, aspects, and advantages of the disclosure will be apparent from a reading of the following.

### Definitions

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%. Numeric values modified by the term “about” include the specific identified value. For example, “about 100” means a number ranging from 95 to 105, including 95, 100, and 105. Recitation of ranges of values herein are merely intended to serve as a shorthand method of referring individually to each separate value falling within the range, unless otherwise indicated herein, and each separate value is incorporated into the specification as if it were individually recited herein.

As used herein, “administration” of a compound or composition to a patient refers to any route of introducing or delivering the compound or composition to a subject. Administration includes self-administration and the administration by another.

As used herein, a “condition,” “disorder,” or “disease” relates to any unhealthy or abnormal state.

As used herein, an “effective amount” or “effective dose” refers to an amount of a compound or composition that treats and/or temporarily prevents, upon single or multiple dose administration, a patient suffering from a disorder, disease, or condition. An effective amount can be determined by the attending diagnostician through the use of known techniques and by observing results obtained under analogous circumstances. In determining the effective amount, a number of factors are considered by the attending diagnostician, including, but not limited to: the species of patient; its size, age, and general health; the specific condition, disorder, or disease involved; the degree of or involvement or the severity of the condition, disorder, or disease, the response of the individual patient; the particular compound administered; the mode of administration; the bioavailability characteristics of the preparation administered; the dose regimen selected; the use of concomitant medication; and other relevant circumstances.

As used herein, a composition is “free” of a component when said component is not intentionally added during manufacture of the composition.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other during a treatment period. Unless specified otherwise, the two or more compounds, agents, or active pharmaceutical ingredients may be administered on different schedules during the treatment period, such as, e.g., with one or more compounds, agents, or active pharmaceutical ingredients being administered once a day and one or more other compounds, agents, or active pharmaceutical ingredients being administered twice a day.

As used herein, the terms “patient,” “subject,” “individual,” and the like, as used herein, are interchangeable and refer to any animal, which may be a human or a non-human animal. Compositions of the invention are particularly suited to use in humans.

As used herein, the terms “prevention” of or “preventing” a disorder, disease, or condition refers to reduction of or reducing the occurrence of the disorder, disease, or condition in a treated sample relative to an untreated control sample, and includes delaying onset, progression, or reduction of severity of one or more symptoms of the disorder or condition relative to the untreated control sample.

As used herein, the term “ocular disorder” refers to any of the signs, symptoms, and/or underlying cause(s) of any physiological disorder that affects the eye or vision.

As used herein, a “pharmaceutically acceptable excipient” refers to a carrier or an excipient that is useful in preparing a pharmaceutical composition. For example, a pharmaceutically acceptable excipient is generally safe and includes carriers and excipients that are generally considered acceptable for mammalian pharmaceutical use. As a non-limiting example, pharmaceutically acceptable excipients may be solid, semi-solid, or liquid materials which, in the aggregate, can serve as a vehicle or medium for active ingredients. Some examples of pharmaceutically acceptable excipients are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, a composition according to the present disclosure is “stable” if the lifitegrast within the composition degrades by less than about 10% after being stored at about 25° C. for 10 weeks, as measured by high-performance liquid chromatography (HPLC) or an equivalent appropriate technique such as that within the U.S. Pharmacopeia.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

As will be understood by one of ordinary skill in the art, each range disclosed herein includes all possible subranges as well as individual numerical values within that range, including endpoints. As a non-limiting example, a range of “0.001% to 0.02%” includes and would be understood to specifically disclose subranges such as “0.004% to 0.01%,” “0.005% to 0.02%,” etc., as well as all individual numbers within the disclosed range, for example, 0.001%, 0.004%, 0.005%, 0.01%, 0.02%, etc.

Claims or descriptions that include “or” or “and/or” between members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

### DETAILED DISCLOSURE

The ophthalmic compositions of the present disclosure comprise (a) lifitegrast and (b) at least one semifluorinated alkane.

In some embodiments, lifitegrast is included in the composition at a concentration from about 0.005% (w/w) to about 5% (w/w), for example, from about 0.02% (w/w) to about 4% (w/w), from about 0.5% (w/w) to about 3% (w/w), or from about 0.75% (w/w) to about 2% (w/w). In some preferred embodiments, the concentration is less than 5.0%, for example about 4.5%, about 4.0%, or about 3.5%.

The ophthalmic compositions of the present disclosure comprise lifitegrast. This is typically included in the form of lifitegrast free acid, but in some embodiments may be included in the form of a pharmaceutically acceptable salt. Where a salt is used, concentrations of lifitegrast disclosed herein are calculated according to the quantity of lifitegrast free acid.

The ophthalmic compositions of the present disclosure comprise at least one semifluorinated alkane (SFA). Semifluorinated alkanes are linear or branched alkanes some of whose hydrogen atoms have been replaced by fluorine. In particular, semifluorinated alkanes are compounds composed of at least one perfluorinated hydrocarbon segment and at least one non-fluorinated hydrocarbon segment. According to a nomenclature used herein, the semifluorinated alkanes may be referred to as FnHm, wherein F refers to the perfluorinated hydrocarbon segment (F-segment), H refers to the non-fluorinated hydrocarbon segment (H-segment), and n and m refer to the number of carbon atoms of those segments, respectively. For example, F3H3 is used for perfluoropropylpropane and F4H5 is used for 1-perfluorobutylpentane. This type of nomenclature is usually used for compounds having linear segments. Therefore, unless otherwise indicated, it should be assumed that F3H3 means 1-perfluoropropylpropane, rather than 2-perfluoropropylpropane, 1-perfluoroisopropylpropane, or 2-perfluoro-isopropylpropane.

In some embodiments, the at least one semifluorinated alkane may have one non-fluorinated hydrocarbon segment attached to one perfluorinated hydrocarbon segment, according to the general formula FnHm. In some embodiments, the at least one semifluorinated alkane is a compound according to the formula FnHm, wherein the F-segment and the H-segment are linear or branched and wherein n and m, which may be the same or different, are independently chosen from 3 to 20 carbon atoms, for example from 3 to 10 carbon atoms, or from 4 to 8 carbon atoms. In some embodiments, the at least one semifluorinated alkane is a compound according to the formula FnHm, wherein the F-segment and the H-segment are linear and wherein n and m, which may be the same or different, are independently chosen from 3 to 20 carbon atoms, for example from 3 to 10 carbon atoms, or from 4 to 8 carbon atoms.

In some embodiments, the ratio of the carbon atoms of the F-segment to the carbon atoms of the H-segment (said ratio obtained by dividing the number of carbon atoms in the F-segment by the number of carbon atoms in the H-segment) of the at least one linear or branched semifluorinated alkane ranges between 0.5 and 3.0, for example between 0.6 and 1.0. For example, the ratio of the carbon atoms of the F-segment to the carbon atoms of the H-segment for 1-perfluorohexyloctane (F6H8) is 0.75. In some embodiments, the at least one semifluorinated alkane is a linear semifluorinated alkane according to the formula FnHm, wherein the ratio of the carbon atoms of the F-segment to the carbon atoms of the H-segment ranges between 0.5 and 3.0, for example between 0.6 and 1.0.

In some embodiments, the at least one semifluorinated alkane may have two perfluorinated hydrocarbon segments separated by one non-fluorinated hydrocarbon segment, according to the general formula FnHmFo, wherein n, m, and o refer to the number of carbon atoms of the first perfluorinated hydrocarbon segment, the non-fluorinated hydrocarbon segment, and the second perfluorinated hydrocarbon segment, respectively. In some embodiments, the at least one semifluorinated alkane is a compound according to the formula FnHmFo, wherein the F-segments and the H-segment are linear or branched and wherein n, m, and o, which may be the same or different, are independently chosen from 3 to 20 carbon atoms, for example from 3 to 10 carbon atoms, or from 4 to 8 carbon atoms.

In some embodiments, the at least one semifluorinated alkane may be a linear semifluorinated alkane comprising a branched non-fluorinated hydrocarbon segment comprising one or more alkyl groups chosen from —CH₃, —C₂H₅, —C₃H₇, —C₄H₉, or any combination thereof. In some embodiments, the at least one semifluorinated alkane may be a linear semifluorinated alkane comprising a branched perfluorinated hydrocarbon segment comprising one or more perfluorinated alkyl groups chosen from —CF₃, —C₂F₅, —C₃F₇, —C₄F₉, or any combination thereof.

In some embodiments, the at least one semifluorinated alkane may comprise a combination of more than one semifluorinated alkane.

In some embodiments, the at least one semifluorinated alkane is chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof. In some embodiments, the at least one semifluorinated alkane is chosen from F4H4, F4H5, F4H6, F5H5, F5H6, F5H7, F5H8, F6H6, F6H7, F6H8, F6H9, F6H10, F8H8, F8H10, F8H12, F10H10, or any combination thereof. In some embodiments, the at least one semifluorinated alkane is chosen from F4H5, F4H6, F5H6, F5H7, F6H6, F6H7, F6H8, or any combination thereof.

In some embodiments, the at least one semifluorinated alkane comprises 1-perfluorohexyl-octane (F6H8). In some embodiments, the at least one semifluorinated alkane consists essentially of 1-perfluorohexyl-octane (F6H8). In some embodiments, the ophthalmic composition consists essentially of: (a) lifitegrast and (b) no more than two semifluorinated alkanes e.g. 1-perfluorohexyl-octane and one other SFA. In some embodiments, the ophthalmic composition consists essentially of: (a) lifitegrast and (b) 1-perfluorohexyl-octane.

In some embodiments, the ophthalmic composition consists essentially of: (a) lifitegrast and (b) one or two perfluorohexyl-octanes. In some embodiments, the ophthalmic composition consists essentially of: (a) lifitegrast (b) 1-perfluorohexyl-octane and (c) 2-perfluorohexyl-octane. Component (a) may be present at up to 5% (w/w) of the composition e.g. from 1-5%. Component (b) may be present at up to 99.5% (w/w) of the composition e.g. from 95-99.5%. Component (c) may be present at up to 2% (w/w) of the composition e.g. from 0.05-1.5%.

The compositions disclosed herein may be used for the treatment or temporary prevention of dry eye disease. They may be useful for treatment of the signs and symptoms of dry eye disease.

In some embodiments, the ophthalmic composition comprises (a) lifitegrast; and (b) at least one semifluorinated alkane chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof.

In some embodiments, the ophthalmic composition comprises (a) lifitegrast; and (b) at least one semifluorinated alkane chosen from F4H5, F4H6, F5H6, F5H7, F6H6, F6H7, F6H8, or any combination thereof.

In some embodiments, the ophthalmic composition comprises (a) lifitegrast; and (b) at least one semifluorinated alkane comprising F6H8.

In some embodiments, the ophthalmic composition comprises (a) lifitegrast; and (b) at least one semifluorinated alkane, wherein the at least one semifluorinated alkane consists essentially of F6H8.

In some embodiments, the ophthalmic composition comprises (a) lifitegrast; and (b) no more than two semifluorinated alkanes.

In embodiments containing “no more than two” or “up to two” semifluorinated alkanes, these may be a single SFA (e.g. 1-perfluorohexyl-octane) or a mixture of two structural isomers of a SFA (e.g. a mixture of 1-perfluorohexyl-octane and 2-perfluorohexyl-octane). In such isomer mixtures, a straight chain SFA (e.g. 1-perfluorohexyl-octane) may be present in excess compared to a branched chain SFA (e.g. 2-perfluorohexyl-octane). For example, the SFA component can consist of 95% (w/w) or more 1-perfluorohexyl-octane with the balance being 2-perfluorohexyl-octane e.g. at least 96%, at least 97%, at least 98%, or at least 99% 1-perfluorohexyl-octane.

In some embodiments, the ophthalmic compositions of the present disclosure are liquid composition. In some embodiments, the ophthalmic compositions of the present disclosure are liquid compositions comprising water. In preferred embodiments, the ophthalmic compositions of the present disclosure are liquid compositions that are substantially water-free (non-aqueous).

In some embodiments, the ophthalmic compositions of the present disclosure are stable liquid compositions. In some embodiments, the ophthalmic compositions of the present disclosure are stable non-aqueous liquid compositions.

In some embodiments, the ophthalmic compositions of the present disclosure may be administered in combination with an effective amount of an additional therapeutic agent. In some embodiments, the ophthalmic compositions of the present disclosure may be administered in combination with an effective amount of at least one additional therapeutic agent chosen from redness reduction agents, NSAIDS, steroids, antifungals, aminopenicillins, macrolides, mite-killing compounds, antihistamines, anti-inflammatory agents, anti-allergens, antibiotics, or any combination thereof.

In some embodiments, the ophthalmic compositions of the present disclosure further comprise, in the same composition, an effective amount of an additional therapeutic agent. In some embodiments, the ophthalmic compositions of the present disclosure further comprise an effective amount of at least one additional therapeutic agent chosen from redness reduction agents, NSAIDS, steroids, antifungals, aminopenicillins, macrolides, mite-killing compounds, antihistamines, anti-inflammatory agents, anti-allergens, antibiotics, or any combination thereof.

In some embodiments, the ophthalmic compositions of the present disclosure may further include at least one additional non-therapeutic component and/or pharmaceutically acceptable excipient, including, but not limited to, tonicity agents, preservatives, buffers, pH adjustors, anti-oxidants, delivery vehicles, stabilizers, suspending agents, viscosity-increasing agents, wetting agents, solubilizing agents, chelating agents, nitrous oxide inhibitors, isotonic agents, humectants, surfactants, and the like. The proportion and nature of any additional non-therapeutic component and/or pharmaceutically acceptable excipient may be determined by the chosen route of administration and standard pharmaceutical practice. One of ordinary skill in the art can readily select the proper form and route of administration depending upon the disorder or condition to be treated, the stage of the disorder or condition, and other relevant circumstances.

In some embodiments, the total lifitegrast degradation products formed in the formulations of the present invention is less than 1.0% when stored at a temperature of 40° C. for a period of one month. In some further embodiments, the total degradation products of the lifitegrast is less than about 0.9%, about 0.8%, about 0.7%, about 0.6%, about 0.5%, about 0.4%, about 0.3%, about 0.2%, or about 0.1% when stored at a temperature of 40° C. for a period of one month.

In some further embodiments, the total degradation products of the lifitegrast formed in the formulations of the present invention is less than 1.0% when stored at a temperature of 40° C. for a period of about two months, about three months, about four months, about five months about six months.

In some cases the pharmaceutically acceptable formulation expires in about 1-5 years. In some cases the formulation expires in about 1, 2, 3 or 4 years. In some cases the formulation expires in more than 5 years. In some cases the formulation expires in less than a year. In some cases the formulation expires in 1, 2, 3, 4, 5, 6, 7, 8, 9, 10, or 11 months.

In some cases the total the lifitegrast degradation products at the time of product expiration are in the range of above 0.1-10%. In some cases the total degradation product at the time of expiration is in the range of about 0.01-1, about 0.01-2, about 0.01-3, about 0.01-4, about 0.01-5, about 0.01-6, about 0.01-7, about 0.01-8, or about 0.01-9, about 1-2, about 1-3, about 1-4, about 1-5, about 1-6, about 1-7, about 1-8, about 1-9, about 2-3, about 3-4, about 2-5, about 2-6, about 2-7, about 2-8, about 2-9, about 3-4, about 3-5, about 3-6, about 3-7, about 3-8, about 3-9, about 3-10, about 4-5, about 4-6, about 4-7, about 4-8, about 4-9, about 4-10, about 5-6, about 5-7, about 5-8, about 5-9, about 5-10, about 6-7, about 6-8, about 6-9, about 6-10, about 7-8, about 7-9, about 7-10, about 8-9, about 8-10 or about 9-10%. In some embodiments the amount of total degradation product at the time of expiration is about 1%, about 2%, about 3%, about 4%, about 5%, about 6%, about 7%, about 8%, about 9%, about 10%. In some embodiments the amount of total degradation product at the time of expiration is about 0.01%, about 0.05%, about 0.1%, about 0.15%, about 0.2%, about 0.25%, about 0.3%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, or about 1.0%.

In some embodiments of the invention the formulations of lifitegrast contain one or more anti-oxidant to prevent oxidative degradation of the lifitegrast. In some embodiments the one or more anti-oxidants comprise a thiosulfate salt. In some embodiments the one or more anti-oxidants used in the formulation of lifitegrast include sodium thiosulfate. In some embodiments the anti-oxidants used in the formulation of lifitegrast include a metabisulfite salt. In some embodiments the anti-oxidants comprise sodium bisulfate.

Other anti-oxidants which can be used to form pharmaceutical formulations of the invention include, but are not limited to, propyl, octyl and dodecyl esters of gallic acid, butylated hydroxyanisole (BHA, usually purchased as a mixture of ortho and meta isomers), green tea extract, uric acid, cysteine, pyruvate, nordihydroguaiaretic acid, ascorbic acid, salts of ascorbic acid such as ascorbyl palmitate and sodium ascorbate, ascorbyl glucosamine, vitamin E (i.e., tocopherols such as α-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), indole-3-carbinol, pycnogenol, melatonin, sulforaphane, pregnenolone, lipoic acid and 4-hydroxy-5-methyl-3 [2H]-furanone. In various embodiments, one or more of the above anti-oxidants are excluded, or are present in less than effective amounts, either alone or in combination.

In some embodiments the amount of anti-oxidants used is in the range of about 0.01-0.5% w/v. In some embodiments the amount of anti-oxidants used is in the range of about 0.1-about 0.5%, about 0.2-about 0.5%, about 0.3-about 0.5%, about 0.4-about 0.5%, about 0.01-about 0.4%, about 0.1-about 0.4%, about 0.2-about 0.4%, about 0.3-about 0.4%, about 0.01-about 0.3%, about 0.1-about 0.3%, about 0.2-about 0.3%, about 0.01-about 0.2%, about 0.1-about 0.2%, or about 0.01-about 0.1%. In some embodiments, sodium thiosulfate is present in an amount to provide anti-oxidant stability to the formulation, and the amount of sodium thiosulfate as a percentage by weight of all total anti-oxidants is greater than 50% by weight.

In some embodiments, vitamin E and/or derivatives of vitamin E are present in an amount to provide anti-oxidant stability to the formulation, and the amount of vitamin E and/or derivatives of vitamin E as a percentage by weight of all total anti-oxidants is greater than 50% by weight.

As noted above, it is also possible to provide pharmaceutical formulations of the invention which are free from anti-oxidants, thus simplifying their preparation and avoiding potential irritation caused by anti-oxidant chemicals. The ability to maintain lifitegrast in stable form in compositions of the invention despite the absence of anti-oxidant is particularly surprising because oxygen is highly soluble in SFAs (and in particular in F6H8). The Xiidra product includes sodium thiosulfate as anti-oxidant.

It is also possible to provide pharmaceutical formulations of the invention which are free from preservatives even when they are provided in a multi-dose container, thus simplifying their preparation and, as discussed above, avoiding potential irritation caused by known preservatives. Without wishing to be bound by theory, it is believed that the SFA can provide a preservative effect in the composition, as well as providing a therapeutic ocular effect.

Preferred compositions of the invention are free from both anti-oxidants and from extrinsic preservatives (i.e. save for the SFA(s), considering these as preservatives). The compositions may also be free from water.

In some cases pharmaceutical formulations of the lifitegrast are stored at −5 to 65° C. In some cases the formulations can be stored at about 0° C., about 5° C., about 10° C., about 15° C., about 20° C., about 25° C., about 30° C., about 35° C., about 40° C., about 45° C., about 50° C., about 55° C., about 60° C., or about 65° C. In various embodiments, the compositions are stored at or below ambient temperature.

In some embodiments, the ophthalmic compositions of the present disclosure are sterile, according to USP/EP criteria. In some embodiments, sterility is conferred by any conventional method. In some embodiments, sterility is conferred by filtration. In some embodiments, sterility is conferred by irradiation. In some embodiments, sterility is conferred by heating. In some embodiments, sterility is conferred by conducting the manufacturing process under aseptic conditions.

Further disclosed herein are methods for the treatment or temporary prevention of at least one ocular disorder comprising administering to a patient in need thereof an effective amount of an ophthalmic composition as described above comprising (a) lifitegrast; and (b) at least one semifluorinated alkane.

The at least one ocular disorder may include, but is not limited to, dry eye disease, dry eye, meibomian gland dysfunction, adult chlamydial ophthalmia, ocular rosacea presbyopia, recalcitrant recurrent corneal erosions, ocular cicatricial pemphigoid, Sjogren's syndrome (SS), non-SS keratoconjunctivitis sicca (KCS), conjunctivitis, allergic conjunctivitis, endophthalmitis, keratitis, uveitis, styes, eye inflammation, eye discomfort or pain, itching of the eye, inflammatory dry eye, redness of the eye, watery eye, stinging or burning of the eye, gritty sensation of the eye, eye sensitivity to light, blurred vision, more frequent blinking, pressure behind the eye, eyelids that are greasy, crusty, itchy, swollen or sticking, and the like.

In some embodiments, the at least one ocular disorder may be chosen from dry eye disease, dry eye, meibomian gland dysfunction, adult chlamydial ophthalmia, ocular rosacea presbyopia, recalcitrant recurrent corneal erosions, ocular cicatricial pemphigoid, Sjogren's syndrome (SS), non-SS keratoconjunctivitis sicca (KCS), conjunctivitis, allergic conjunctivitis, endophthalmitis, keratitis, uveitis, or any combination thereof.

In some embodiments, the at least one ocular disorder may be chosen from conjunctivitis, keratitis, dry eye disease, or any combination thereof.

In some embodiments, the at least one ocular disorder is dry eye disease. In some embodiments, the dry eye disease is aqueous deficient dry eye disease. In other embodiments, it is evaporative dry eye disease. In yet further embodiments, it is both aqueous deficient dry eye disease and evaporative dry eye disease.

In some embodiments, the dry eye disease has hyperosmolarity as a cause and/or as a symptom. In some embodiments, the dry eye disease has tear film instability as a cause and/or as a symptom. In some embodiments, the dry eye disease has both hyperosmolarity and tear film instability as a cause and/or as a symptom.

In some embodiments, the methods of the present disclosure comprise treatment or temporary prevention of dry eye disease comprising administering to a patient in need thereof an effective amount of an ophthalmic composition as described above comprising (a) lifitegrast; and (b) at least one semifluorinated alkane.

In some embodiments, the methods of the present disclosure comprise treatment or temporary prevention of dry eye disease comprising administering to a patient in need thereof an effective amount of an ophthalmic composition comprising (a) lifitegrast; and (b) at least one semifluorinated alkane chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof.

In some embodiments, the methods of the present disclosure comprise treatment or temporary prevention of dry eye disease comprising administering to a patient in need thereof an effective amount of an ophthalmic composition comprising (a) lifitegrast; and (b) at least one semifluorinated alkane chosen from F4H5, F4H6, F5H6, F5H7, F6H6, F6H7, F6H8, or any combination thereof.

In some embodiments, the methods of the present disclosure comprise treatment or temporary prevention of dry eye disease comprising administering to a patient in need thereof an effective amount of an ophthalmic composition comprising (a) lifitegrast; and (b) at least one semifluorinated alkane chosen from F4H5, F4H6, F5H6, F5H7, F6H6, F6H7, F6H8, or any combination thereof.

In some embodiments, the methods of the present disclosure comprise treatment or temporary prevention of dry eye disease comprising administering to a patient in need thereof an effective amount of an ophthalmic composition comprising (a) lifitegrast; and (b) at least one semifluorinated alkane comprising F6H8.

In some embodiments, the methods of the present disclosure comprise treatment or temporary prevention of dry eye disease comprising administering to a patient in need thereof an effective amount of an ophthalmic composition comprising (a) lifitegrast; and (b) at least one semifluorinated alkane, wherein the at least one semifluorinated alkane consists essentially of F6H8.

Where the composition of the invention is a suspension it should be redispersed prior to use. Thus a method may include, before a step of administering, a step of redispersing lifitegrast in the liquid phase (e.g. the SFA). For instance, a container closure system such as a bottle may be shaken (or inverted several times) to achieve redispersal, and droplets of the suspension may then be dispensed. Product information included with a composition may include a written statement informing a subject to perform this redispersal (e.g. to shake before use), or informing a medical professional to provide such advice to a subject.

Where a subject wears contact lenses, it may be desirable that these are removed prior to administration of a composition of the invention to the subject's eye(s). Thus a method may include, before a step of administering, a step of contact lens removal. Re-fitting of the contact lenses may be delayed e.g. for at least 30 minutes after administration of the composition. Product information included with a composition may include a written statement informing a subject to perform this contact lens removal and to delay re-fitting, or informing a medical professional to provide such advice to a subject.

In some embodiments, the ophthalmic composition further comprises a preservative. In some embodiments, the preservative is selected from benzalkonium chloride, cetrimonium, sodium perborate, stabilized oxychloro complex SofZia, polyquaternium-1, chlorobutanol, edetate disodium, polyhexamethylene biguanide, or combinations thereof.

In some embodiments, the ophthalmic composition further comprises a buffer agent. In some embodiments, the buffer agent is selected from borates, borate-polyol complexes, phosphate buffering agents, citrate buffering agents, acetate buffering agents, carbonate buffering agents, organic buffering agents, amino acid buffering agents, or combinations thereof.

In some embodiments, the ophthalmic composition comprises the lifitegrast and SFA components, and an oil. The oil should be miscible with the SFA component(s). For instance, the oil can be a mineral oil (such as light mineral oil) or triglycerides (such as medium-chain triglycerides) or a tocopherol (such as α-tocopherol). Thus a composition can consist of up to two SFAs (e.g. F6H8), lifitegrast (e.g. free acid), and an oil component (e.g. a mineral oil or triglycerides or a tocopherol).

In some embodiments, the ophthalmic compositions can be administered by any convenient route. In some embodiments, the ophthalmic compositions are any liquid form suitable for topical application. In some embodiments, the ophthalmic compositions are topically administered to the ocular surface. In some embodiments, the ophthalmic compositions are topically administered to the cornea. In some embodiments, the ophthalmic compositions are instilled into the conjunctival sac.

In some embodiments, the composition may be in the form of a liquid (such as a suspension, a solution, an emulsion, or the like) or in the form of an ointment. In some embodiments, the ophthalmic composition may be in the form of a suspension. In some embodiments, the ophthalmic composition may be in the form of a solution. In some embodiments the composition may be administered as an eye drop. In some embodiments, the ophthalmic composition may be in the form of a suspension that may be administered as an eye drop. In some embodiments, the ophthalmic composition may be in the form of a solution that may be administered as an eye drop. In some embodiments, the ophthalmic composition may be in the form of an ointment. In some embodiments, the ophthalmic composition may be administered as a spray. In some embodiments, the ophthalmic composition may be administered as artificial tears. In some embodiments, the ophthalmic composition may be in the form of a contact lens adsorbent.

Administration as eye drops is typical for the treatment or temporary prevention of dry eye disease, with one or more drops of the composition being applied to an eye of the subject. Droplets may have a volume of 25 μL or lower, and can be as small as 8-15 μL e.g. between 11-12 μL. These droplet volumes are smaller than present in the Xiidra product (35 μL) and lead to lower nasolacrimal drainage, thus reducing sensations of bad taste.

After application of eye drops, it can be useful to use nasolacrimal compression, obstruction, or occlusion (for example by applying compression to the lacrimal sac at the medial canthus). This procedure can reduce the amount of liquid entering the nasolacrimal duct and draining into the throat where they can lead to sensations of bad taste.

Proper dosages of the ophthalmic compositions of the present disclosure are concentration-dependent. To determine the specific dose, a skilled artisan would have to take into account kinetics and absorption characteristics of the particular ingredients. In addition, the dosage may be dependent on the route of administration. The dosages may also be dependent on the degree of antibacterial and/or anti-inflammatory effect desired in a patient.

Methods of treating comprise administration of a daily dose of an ophthalmic composition of the present disclosure to a subject in need thereof. Determining and adjusting an appropriate dosing regimen (e.g., adjusting the number of doses and frequency of dosing) per day can be performed by one of ordinary skill in the relevant art, and will depend upon various factors such as the nature and progression of a disorder and/or condition associated with allergies and eye redness, the health, and/or age of the subject. In some embodiments, the ophthalmic compositions of the present disclosure are administered to a patient in a single dose per day, in two doses per day, in three doses per day, in four doses per day, etc. or up to, for example, ten doses per day. In some embodiments, the composition is administered to a patient for at least one week, at least two weeks, at least three weeks, at least one month, at least 2 months, at least 3 months, at least 4 months, at least 5 months, at least 6 months, at least 12 months, at least one year, or for more than one year. When using eye drops, one dose is typically a single droplet per eye.

Further disclosed herein are container closure systems containing an ophthalmic composition as described above comprising (a) lifitegrast and (b) at least one semifluorinated alkane.

The ophthalmic compositions disclosed herein may be kept in container closure systems (e.g., vials, ampoules, bottles, tubes, syringes, dispenser packages, or other suitable containers). In some embodiments, the compositions may be packaged either in a single-dose unit container closure system or multi-dose container closure system. In some embodiments, the compositions may be packaged in a single-dose unit container closure system. Container closure systems may be composed of one or more materials appropriate for its use, such as aluminum, glass, polypropylene, polyethylene (e.g., low-density polyethylene and high-density polyethylene), polyethylene terephthalate, and polyethylene terephthalate glycol. Plastic container closure systems weigh less, are more resistant to shock and other mechanical influences, cost less, and offer more design possibilities than glass. Polyethylene, e.g., low-density polyethylene (LDPE), without or with additives, and polypropylene (PP) are the plastics required by the European Pharmacopoeia. In some embodiments, the container closure system comprises polypropylene. In some embodiments, the container closure system is composed of polypropylene (e.g. a polypropylene bottle). In some embodiments, the container closure systems may be squeezable to allow delivery of the ophthalmic composition as drops to the eye (e.g. squeezable by hand). A container closure system may thus have a droplet-forming outlet. This outlet can facilitate dropwise dispensing of a composition from the container. The outlet can be protected by a screw cap when not in use. Thus the ophthalmic composition is located in a polypropylene bottle (e.g. a translucent or transparent polypropylene bottle) which can be squeezed by hand to expel the ophthalmic composition dropwise through an outlet.

In some embodiments, a single container closure system can comprise one or more compartments for containing a provided ophthalmic composition, and/or appropriate carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated.

Ophthalmic compositions disclosed herein may be prepared according to any known method for the manufacture of ophthalmic formulations or preparations. As will be appreciated by those of ordinary skill in the art, a number of methods are known. In some embodiments, the ophthalmic compositions disclosed herein may be prepared by any conventional technique, such as, e.g., those described in Remington: The Science and Practice of Pharmacy, 21st edition, 2005, ed. D. B. Troy, Lippincott Williams & Wilkins, Philadelphia (or 23rd edition 2020, ed. A. Adejare), and Encyclopedia of Pharmaceutical Technology, eds. J. Swarbrick and J. C. Boylan, 1988-1999, Marcel Dekker, New York (or 4th edition 2013, ed. J. Swarbrick).

In some embodiments, the pharmaceutical composition further comprises a surfactant to disperse insoluble ingredients or to improve solubilization. Numerous ocular surfactants are disclosed in US2019/0328773, which is incorporated herein by reference. Several of those are listed below. Additional surfactants and relevant ranges are listed in US2018/0221407A1, which is incorporated herein by reference.

The composition can further include one or more co-solubilizers such as a surfactant. The surfactant may vary, and may include any compound that is surface active or can form micelles. A surfactant may be used for assisting in dissolving an excipient or an active agent, dispersing a solid or liquid in a composition, enhancing wetting, modifying drop size, stabilizing an emulsion, or a number of other purposes. Examples of surfactants may include, but are not limited to, surfactants of the following classes: alcohols, for example polyvinyl alcohol; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal & veg.); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants such as Soluplus® (from BASF); propoxylated & ethoxylated fatty acids, alcohols, or alkyl phenols; protein-based surfactants; sarcosine derivatives; sorbitan derivatives; sucrose and glucose esters and derivatives; and saponins. In some embodiments, the surfactant may include polyethylene glycol (15)-hydroxystearate (CAS Number 70142-34-6, available as SOLUTOL HS 15@ from BASF), a polyoxyethylene-polyoxypropylene block copolymer (CAS No. 9003 November 6, available as PLURONIC® F-68 from BASF), polyoxyethylene 40 stearate (POE40 stearate), polysorbate 80 or polyoxyethylene (80) sorbitan monooleate (CAS No. 9005-65-6), sorbitan monostearate (CAS No. 1338-41-6, available as SPAN™ 60 from Croda International PLC), or polyoxyethyleneglyceroltriricinoleate 35 (CAS No. 61791 December 6, available as CREMOPHOR EL® from BASF), ethoxylated castor oil, such as Cremophor EL (CAS Number 61791 December 6). Suitable cosolubilizers include, but are not limited to, povidone, and acrylates (e.g. PEMULEN®).

In some implementations, the surfactant is a non-ionic surfactant that can in some instances include polyoxyethylene sorbitan monooleate (Polysorbate-80) represented by CAS No. 9005-65-6, such as Tween® 80, available from Sigma-Aldrich. In some implementations, the non-ionic surfactant includes polyoxyethylene lauryl ether represented by CAS No. 9002-92-0, such as Brij® 35, available from Sigma-Aldrich. In some implementations, the non-ionic surfactant polyol includes polyethylene glycol)-Wock-poly (propylene glycol)-Wock-poly(ethylene glycol) represented by CAS No. 9003 November 6, such as Pluronic™ F-127, available from BASF SE. Other non-ionic surfactants are considered herein including, but not limited to ethoxylates, fatty alcohol ethoxylates, alkylphenol ethoxylates, fatty acid ethoxylates, special ethoxylated fatty esters and oils, ethoxylated amines and/or fatty acid amides, terminally blocked ethoxylates, fatty acid esters of polyhydroxy compounds, fatty acid esters of glycerol, fatty acid esters of sorbitol, Tweens, fatty acid esters of sucrose, alkyl polyglucosides, amine oxides, sulfoxides, phosphine oxides.

The amount of surfactant may vary. In some implementations, the surfactant can be used at a concentration from about 0.05% w/v to about 5.0% w/v, preferably 0.05% w/v to about 0.5% w/w.

The surfactant incorporated in the compositions is not limited by class. Various classes of surfactants can be incorporated including, but not limited to anionic, cationic, zwitterionic, and nonionic surfactants. It should also be appreciated that multiple combinations of surfactants can be included.

In some embodiments, the ophthalmic compositions of the present disclosure are in polypropylene (PP) container closure systems (e.g., vials, ampoules, bottles, tubes, syringes, dispenser packages, or other suitable containers). Exemplary PP container closure systems include white PP bottles and natural PP bottles. PP containers are squeezable but also are semi-permeable to volatile compounds. It may be desirable for an ophthalmic composition to demonstrate stability in multiple types of container closure systems, each of a different material type.

Where an ophthalmic composition is provided in a multi-dose container closure system, such as a PP bottle, the volume of the ophthalmic composition will typically be large enough to provide at least 10 doses e.g. to provide at least 10 droplets, such as at least 100 droplets. The volume of the composition may be at least 0.5 mL e.g. ≥1 mL or ≥1.5 mL or more. For instance, a PP bottle may contain at least 2 mL of the composition.

The compositions provided herein may optionally include an effective amount of a taste masking agent. In some embodiments, formulations of lifitegrast do not contain a taste-masking agent. The taste-masking agent is one or more agents or compounds which, optionally together, successfully mask or cover the (potential) unpleasant taste of one or more components of the compositions provided herein when present in an effective amount. In some embodiments, the compositions comprise two or more taste masking agents, such as a polyol sweetener and a high intensity sweetener. In some embodiments, the compositions include only a single taste masking agent in the absence of any other sweeteners, flavorants, or taste masking agents. In some embodiments, the taste masking agent is (tri) sodium citrate, sodium citrate, sodium chloride, sodium bicarbonate, and combinations thereof. In some embodiments, the taste masking agent is a polyol sweetener. A specific example of one category of polyol sweeteners include sugars, in particular a sugar selected from the group consisting of dextrose, sucrose, maltose, fructose, lactose, and combinations thereof. Another specific example of another category of polyol sweeteners include sugar alcohols, in particular sugar alcohols selected from the group consisting of xylitol, sorbitol, mannitol, maltitol, isomaltol, isomalt, erythritol, lactitol, maltodextrin, hydrogenated starch hydrolysates, D-xylose, trehalose, and combinations thereof. In some embodiments, the taste masking agent is a high intensity sweetener or a flavor. Useful high intensity sweeteners may be selected from the group consisting of sucralose, neotame, aspartame, salts of acesulfame in particular the potassium salt of acesulfame (acesulfame K), alitame, saccharin and its salts, cyclamic acid and its salts, glycyrrhizin, dihydrochalcones e.g. NHDC, thaumatin, monellin, stevioside, Twinsweet (aspartame-acesulfame salt), and combinations thereof. Still other examples of suitable taste masking agents include salts of gluconate, such as sodium gluconate.

In some embodiments, the taste-masking agent in the compositions is sucralose (e.g. in the absence of other sweeteners, flavorants or taste masking agents). In some embodiments, the taste-masking agent may be present in the composition in an amount of from about 0.01% w/v to about 1.00% w/v. In some embodiments, the compositions provided herein have a concentration of the taste-masking agent from about 0.01% w/v to about 0.05% w/v, from about 0.05% w/v to about 0.10% w/v, from about 0.10% w/v to about 0.50% w/v, from about 0.50% w/v to about 1.00% w/v, or more than 1.00% w/v. In some embodiments, the compositions provided herein have about 0.02% w/v, about 0.05% w/v, about 0.10% w/v, about 0.20% w/v, or about 0.30% w/v of the taste-masking agent.

In some embodiments, the present disclosure provides a container closure system containing an ophthalmic composition comprising (a) lifitegrast and (b) at least one semifluorinated alkane as disclosed above, wherein the container closure system is a PP container closure system.

The present disclosure also provides a kit comprising a formulation container containing an ophthalmic composition comprising (a) lifitegrast and (b) at least one semifluorinated alkane as disclosed above, wherein the container closure system is a PP container closure system. In some embodiments, kits can include a second container (the solvent container) comprising a suitable delivery vehicle for dilution or suspension of the provided ophthalmic composition in the first container (the formulation container) for preparation of administration to a subject. In some embodiments, the contents of the formulation container and contents of the solvent container combine to form at least one unit dosage form. In some embodiments, the solvent container contains a delivery vehicle that is aqueous. In some embodiments, the solvent container contains a delivery vehicle that is non-aqueous. Suitable delivery vehicle for the solvent container are detailed above.

In some embodiments, a single container can comprise one or more compartments for containing a provided ophthalmic composition, and/or appropriate vehicle carrier for suspension or dilution. In some embodiments, a single container can be appropriate for modification such that the container can receive a physical modification so as to allow combination of compartments and/or components of individual compartments. For example, a foil or plastic bag can comprise two or more compartments separated by a perforated seal which can be broken so as to allow combination of contents of two individual compartments once the signal to break the seal is generated. A kit can thus comprise such multi-compartment containers including the ophthalmic compositions disclosed herein and an appropriate solvent and/or an appropriate vehicle carrier for suspension.

### Examples

### F6H8 Preservative Activity

The antimicrobial properties of F6H8 were evaluated according to the Ph. Eur. 5.1.3 method. Results were as follows:

| Organism | Time | Count | Log reduction |
|---|---|---|---|
| S. aureus | Inoculum | 2 × 10⁵ | — |
| | 0 hour | 16 | 4.1 |
| | 6 hours | 0 | 5.3 |
| | 24 hours | 0 | 5.3 |
| | 28 days | 0 | 5.3 |
| P. aeruginosa | Inoculum | 8 × 10⁵ | — |
| | 0 hour | 5 × 10⁴ | 1.2 |
| | 6 hours | 4 × 10² | 3.3 |
| | 24 hours | 8 | 5.0 |
| | 28 days | 0 | 5.9 |
| E. coli | Inoculum | 2 × 10⁵ | — |
| | 0 hour | 2 × 10⁴ | 1.0 |
| | 6 hours | 0 | 5.3 |
| | 24 hours | 0 | 5.3 |
| | 28 days | 0 | 5.3 |
| C. albicans | Inoculum | 9 × 10⁵ | — |
| | 0 hour | 5 × 10³ | 2.3 |
| | 7 days | 0 | 6.0 |
| | 28 days | 0 | 6.0 |
| A. brasiliensis | Inoculum | 7 × 10⁵ | — |
| | 0 hour | 8 × 10⁴ | 0.9 |
| | 7 days | 1 × 10³ | 2.8 |
| | 28 days | 2 × 10³ | 2.5 |

F6H8 meets the acceptance criteria for this assay, namely that (a) the concentrations of viable bacteria show not less than a 2 log reduction from the initial calculated count at 6 hours, not less than a 3 log reduction at 24 hours, and no recovery at 28 days and (b) the concentrations of yeasts and molds show not less than a 2 log reduction from the initial calculated count at 7 days, with no increase at 28 days. Thus F6H8 meets the antimicrobial effectiveness requirements without the need for extrinsic preservative.

This result can be explained by the lack of water activity in the F6H8 liquid. Addition of solid lifitegrast introduces no water activity and so this mixture also will not support microbial growth.

### Suspensions

Lifitegrast free acid was added to F6H8 in various proportions, including (w/w) 0.25%, 1%, 2%, 2.5%, 4%, 5%, and 10%. The lifitegrast is in soluble in F6H8 and forms a suspension. When left standing the lifitegrast settles to form a sediment.

Lifitegrast is stable in the suspensions. During storage at 40° C. levels of lifitegrast in a 4% suspension (i.e. 96% (w/w) F6H8, 4% (w/w) lifitegrast) did not change:

| Time (months) | 0 | 1 | 3 |
|---|---|---|---|
| Lifitegrast (%) | 99.87 | 99.87 | 99.89 |

Similar results were seen in a 2% suspension. No significant impurities were seen when stored at 25° C. for 3 months. A minor impurity increase was seen after 3 months at 40° C., but it was insignificant. Thus lifitegrast and F6H8 are chemically compatible. The same compatibility can be expected with other SFAs as they do not include any additional reactive groups.

Lifitegrast/F6H8 suspensions were supplemented with α-tocopherol or tocopheryl acetate as anti-oxidants (e.g. 2% lifitegrast, 93% F6H8, 5% tocopheryl acetate). After storage for 1 month at 40° C. levels of lifitegrast-derived impurities showed no significant differences with or without the anti-oxidants (e.g. for a 2% emulsion with 5% tocopheryl acetate the level of lifitegrast was 99.92% at time zero and still after 3 months at 40° C.). Oxidation-derived degradation products were not detected with or without anti-oxidant. Lifitegrast and F6H8 are therefore compatible, without needing addition of any anti-oxidant.

Suspensions with 2% or 5% lifitegrast were subjected to centrifugation for 1 hour at 500, 750 or 1000 rpm to accelerate settling of the lifitegrast powder, and the ease and efficiency of redispersibility were evaluated by shaking the settled product by hand (conditions to mimic patient use). After 500 rpm centrifugation the powder could be resuspensed by one gentle inversion. After 750 or 1000 rpm centrifugation resuspension required several gentle inversions or shaking by hand 2 or 3 times. Thus the suspensions show redispersibility which is acceptable for patient use.

Suspensions were filled into polypropylene bottles with a dropper outlet and droplets were weighed. Droplets of F6H8 alone had a mass of 15.91 mg with a standard deviation of 0.55 mg, with a volume of 11.35 μL. Droplets of the 5% lifitegrast suspension weighed 15.93 mg with a standard deviation of 0.43 mg, with a volume of 11.85 μL. Droplets of the suspensions thus show acceptable dose uniformity within a bottle, and lifitegrast does not interfere with the ability of F6H8 to form droplets.

Droplets of the F6H8 suspension are ˜3× smaller than the aqueous droplets of Xiidra™ (5% lifitegrast solution), and so the amount of lifitegrast delivered by a single droplet of 5% suspension is lower than the amount delivered by a single Xiidra™ droplet.

### Ocular Irritation Assessment

The ocular tolerability of lifitegrast/F6H8 suspensions (5%) were assessed, including evaluation of sting/burn reactions relative to the marketed lifitegrast solution (Xiidra™) and the marketed F6H8 product (Miebo™). NZW rabbits (3 per group) received topical ocular doses QID for 5 Days (40 μL/dose=160 μL/eye/day). Ocular irritation was assessed by (a) macroscopic examination (Draize scoring) after the fourth daily dose and, if irritation was noted, again in the following morning and (b) microscopic examination by slit lamp and indirect ophthalmoscopy at days 0 & 5. Sting/burn assessments were taken after the second and fourth daily dose, recording the cumulative duration of blinking/squinting and also noting other indicators (e.g., rubbing).

The suspensions were well tolerated with no toxicologically significant findings after 5 days of dosing.

The maximum weighted score for irritation in any animal was scored as 4 out of a maximum of 110. Suspensions were well tolerated and any negative effects (redness, discharge, conjunctival congestion) were short-lived and did not increase in severity over the duration of the study.

### Epicorneal Tissue Model

The EpiCorneal 3D human tissue model (MatTek) provides a predictive non-animal model for assessing ophthalmic drug delivery. This model was used to assess the effect of F6H8 on lifitegrast permeability and the dose-dependence of permeability characteristics.

Seven formulations were tested: four suspensions (0.25%, 1%, 2.5% and 5%), a second 1% suspension, and two aqueous solutions (1% and 5%). 12 μL volumes were administered to the model and assessed for permeability (collecting 200 μL samples from the receiver at 15, 30, 60, 120, 180 and 240 minutes to measure penetrated lifitegrast concentration) and for lifitegrast concentration in EpiCorneal tissue after 240 minutes.

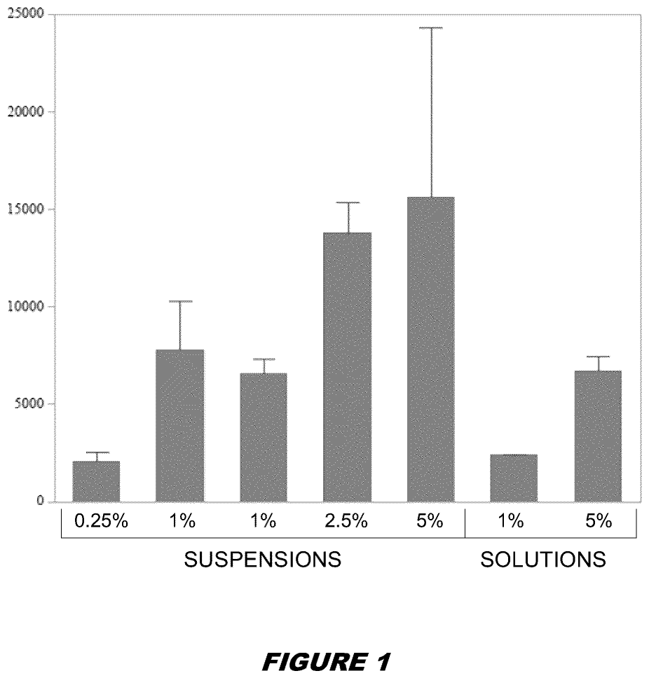

Suspensions had lower lifitegrast permeability than solutions but higher tissue accumulation. A dose-dependent increase in tissue accumulation was observed from 0.25% to 2.5% with the suspensions. FIG. 1 shows cumulative lifitegrast concentrations in EpiCorneal tissue after 4 hours, indicating that the two 1% suspensions provided about the same performance as a 5% aqueous solution, and the 5% suspension provided higher tissue accumulation than the 5% solution. Slower permeation into the receiver using the suspensions might be explained by the need for the solid lifitegrast to dissolve before it can permeate.

### Ocular Pharmacokinetic Study

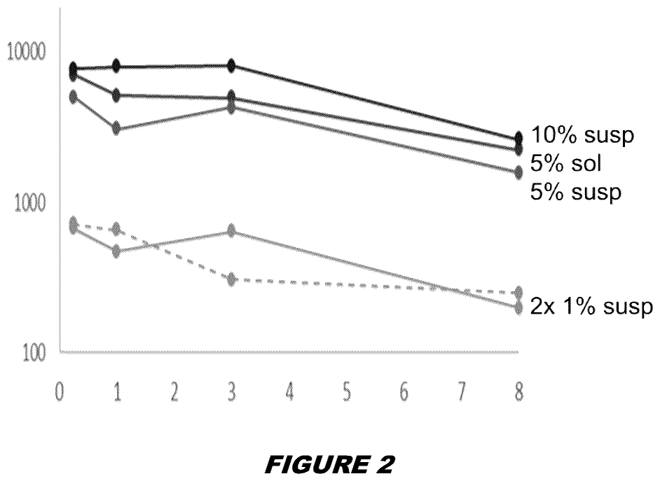

An in vivo pharmacokinetic study was used to evaluate dose-dependent ocular bioavailability of lifitegrast/F6H8 suspensions in dutch belted rabbits. Four suspensions were tested (10%, 5%, and 2× 1%) and compared to a 5% aqueous solution. The dosage volume per eye was 12 μL for the suspensions and 35 μL for the solution. Samples of cornea, bulbar conjunctiva, aqueous humor, anterior sclera, and palpebra conjunctiva were taken after 0.25, 1, 3, and 8 hours. Corneal results are shown in FIG. 2.

Dose-proportional ocular exposure of lifitegrast was seen with suspensions from 1-5%, with a higher than proportional increase in exposure at 10%.

Areas under the lifitegrast concentration-time curve from 0 to 8 hours (AUC_{0-8 hr}) were calculated with linear trapezoidal linear interpolation method using WinNonlin. Values were as follows, relative to the aqueous solution (100%):

| | 5% solⁿ | 1% suspⁿ | 1% suspⁿ | 5% suspⁿ | 10% suspⁿ |
|---|---|---|---|---|---|
| Cornea | 100 | 11 | 9 | 77 | 148 |
| B. conj | 100 | 36 | 28 | 212 | 642 |
| P. conj | 100 | 27 | 17 | 202 | 552 |
| Aq humor | 100 | 14 | 13 | 55 | 309 |
| Sclera | 100 | 13 | 15 | 60 | 195 |

The 5% suspension dose at 12 μL gave the most similar ocular exposure to 5% solution dosed at 35 μL. Thus the administered amount of lifitegrast can be reduced 3-fold while achieving the same ocular exposure.

### Numbered Embodiments

Without limitation, some embodiments of the disclosure include:

### Exemplary Composition Embodiments

Embodiment 1. An ophthalmic composition comprising: (a) lifitegrast and (b) at least one semifluorinated alkane.

Embodiment 2. An ophthalmic composition consisting essentially of: (a) lifitegrast and (b) at least one semifluorinated alkane.

Embodiment 3. The ophthalmic composition according to any one of Embodiments 1-2, wherein the lifitegrast is included in the composition at a concentration from about 0.005% (w/w) to about 5% (w/w), for example, from about 0.02% (w/w) to about 4% (w/w), from about 0.5% (w/w) to about 3% (w/w), or from about 0.75% (w/w) to about 2% (w/w).

Embodiment 4. The ophthalmic composition according to any one of Embodiments 1-3, wherein the at least one semifluorinated alkane is a compound composed of at least one perfluorinated hydrocarbon segment (F-segment) and at least one non-fluorinated hydrocarbon segment (H-segment).

Embodiment 5. The ophthalmic composition according to any one of Embodiments 1-4, wherein the at least one semifluorinated alkane is a compound composed of F-segments and H-segments according to the formula FnHm, wherein the F-segments and the H-segments are linear or branched and wherein n and m, which may be the same or different, are independently chosen from 3 to 20 carbon atoms, or from 3 to 10 carbon atoms, or from 4 to 8 carbon atoms.

Embodiment 6. The ophthalmic composition according to any one of Embodiments 1-5, wherein the at least one semifluorinated alkane is a compound composed of F-segments and H-segments according to the formula FnHm, wherein the F-segments and the H-segments are linear and wherein n and m, which may be the same or different, are independently chosen from 3 to 20 carbon atoms, or from 3 to 10 carbon atoms, or from 4 to 8 carbon atoms.

Embodiment 7. The ophthalmic composition according to any one of Embodiments 1-6, wherein the at least one semifluorinated alkane is a compound composed of F-segments and H-segments according to the formula FnHmFo, wherein the F-segments and H-segments are linear or branched and wherein n, m, and o, which may be the same or different, are independently chosen from 3 to 20 carbon atoms, or from 3 to 10 carbon atoms, or from 4 to 8 carbon atoms.

Embodiment 8. The ophthalmic composition according to any one of Embodiments 1-7, wherein the ratio of the carbon atoms of the F-segment to the carbon atoms of the H-segment of the at least one linear or branched semifluorinated alkane ranges between 0.6 and 3.0 or between 0.6 and 1.0.

Embodiment 9. The ophthalmic composition according to any one of Embodiments 1-8, wherein the ratio of the carbon atoms of the F-segment to the carbon atoms of the H-segment of the at least one linear semifluorinated alkane ranges between 0.6 and 3.0 or between 0.6 and 1.0.

Embodiment 10. The ophthalmic composition according to any one of Embodiments 1-9, wherein the at least one semifluorinated alkane is chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof.

Embodiment 11. The ophthalmic composition according to any one of Embodiments 1-10, wherein the at least one semifluorinated alkane is chosen from F4H4, F4H5, F4H6, F5H5, F5H6, F5H7, F5H8, F6H6, F6H7, F6H8, F6H9, F6H10, F8H8, F8H10, F8H12, F10H10, or any combination thereof.

Embodiment 12. The ophthalmic composition according to any one of Embodiments 1-11, wherein the at least one semifluorinated alkane is chosen from F4H5, F4H6, F5H6, F5H7, F6H6, F6H7, F6H8, or any combination thereof.

Embodiment 13. The ophthalmic composition according to any one of Embodiments 1-12, wherein the at least one semifluorinated alkane comprises 1-perfluorohexyl-octane (F6H8).

Embodiment 14. The ophthalmic composition according to any one of Embodiments 1-13, wherein the at least one semifluorinated alkane consists essentially of 1-perfluorohexyl-octane (F6H8).

Embodiment 15. The ophthalmic composition according to any one of Embodiments 1-14, wherein the at least one semifluorinated alkane comprises perfluorobutylpentane (F4H5).

Embodiment 16. The ophthalmic composition according to Embodiment 15, wherein the at least one semifluorinated alkane consists essentially of perfluorobutylpentane (F4H5).

Embodiment 17. The ophthalmic composition according to any one of Embodiments 1-16, wherein the composition is liquid.

Embodiment 18. The ophthalmic composition according to any one of Embodiments 1-17, wherein the composition is non-aqueous.

Embodiment 19. The ophthalmic composition according to any one of Embodiments 1-18, wherein the composition is in the form of a suspension, a solution, or an emulsion.

Embodiment 20. The ophthalmic composition according to any one of Embodiments 1-19, wherein the composition is in the form of a suspension.

Embodiment 21. The ophthalmic composition according to any one of Embodiments 1-20, wherein the composition is stable.

Embodiment 22. The ophthalmic composition according to any one of Embodiments 1 and 3-21, wherein the composition further comprises at least one non-therapeutic component and/or pharmaceutically acceptable excipient chosen from tonicity agents, preservatives, buffers, pH adjustors, anti-oxidants, delivery vehicles, stabilizers, suspending agents, viscosity-increasing agents, wetting agents, solubilizing agents, chelating agents, nitrous oxide inhibitors, isotonic agents, humectants, surfactants, or any combination thereof.

Embodiment 23. The ophthalmic composition according to any one of Embodiments 1-22, wherein the composition is substantially free of an anti-oxidant.

Embodiment 24. The ophthalmic composition according to any one of Embodiments 1-23, wherein the composition is substantially free of a preservative. Exemplary Method of Treatment Embodiments:

Embodiment 25. A method for the treatment or temporary prevention of at least one ocular disorder comprising administering to a patient in need thereof an effective amount of an ophthalmic composition comprising (a) lifitegrast and (b) at least one semifluorinated alkane.

Embodiment 26. The method according to Embodiment 25, wherein the ophthalmic composition is the ophthalmic composition according to any of Embodiments 1-24.

Embodiment 27. The method according to any one of Embodiments 25-26, wherein the at least one ocular disorder is chosen from dry eye disease, dry eye, meibomian gland dysfunction, adult chlamydial ophthalmia, ocular rosacea presbyopia, recalcitrant recurrent corneal erosions, ocular cicatricial pemphigoid, Sjogren's syndrome (SS), non-SS keratoconjunctivitis sicca (KCS), conjunctivitis, allergic conjunctivitis, endophthalmitis, keratitis, uveitis, styes, eye inflammation, eye discomfort or pain, itching of the eye, inflammatory dry eye, redness of the eye, watery eye, stinging or burning of the eye, gritty sensation of the eye, eye sensitivity to light, blurred vision, more frequent blinking, pressure behind the eye, eyelids that are greasy, crusty, itchy, swollen or sticking, or any combination thereof.

Embodiment 28. The method according to any one of Embodiments 25-27, wherein the at least one ocular disorder is chosen from dry eye disease, dry eye, meibomian gland dysfunction, adult chlamydial ophthalmia, ocular rosacea presbyopia, recalcitrant recurrent corneal erosions, ocular cicatricial pemphigoid, Sjogren's syndrome (SS), non-SS keratoconjunctivitis sicca (KCS), conjunctivitis, allergic conjunctivitis, endophthalmitis, keratitis, uveitis, or any combination thereof.

Embodiment 29. The method according to any one of Embodiments 25-28, wherein the at least one ocular disorder is chosen from conjunctivitis, keratitis, dry eye disease, or any combination thereof.

Embodiment 30. A method for the treatment or temporary prevention of dry eye disease comprising administering to a patient in need thereof an effective amount of an ophthalmic composition comprising (a) lifitegrast and (b) at least one semifluorinated alkane.

Embodiment 31. The method according to Embodiment 30, wherein the ophthalmic composition is the ophthalmic composition according to any of Embodiments 1-24.

Embodiment 32. The method according to any one of Embodiments 25-31, wherein the ophthalmic composition is topically administered to the ocular surface of the patient, or topically administered to the cornea of the patient, or instilled into the conjunctival sac of the patient.

Embodiment 33. The method according to any one of Embodiments 25-32, wherein the ophthalmic composition is administered in the form of a suspension, an ointment, a solution, or a spray.

Embodiment 34. The method according to any one of Embodiments 25-33, wherein the ophthalmic composition is administered in the form of a suspension that may be administered as an eye drop.

Embodiment 35. The method according to any one of Embodiments 25-34, wherein the ophthalmic composition is administered to the patient in a single dose per day, in two doses per day, in three doses per day, in four doses per day, or in up to ten doses per day.

Embodiment 36. The method according to any one of Embodiments 25-35, wherein the ophthalmic composition is administered to the patient for at least one week, at least two weeks, at least three weeks, at least one month, at least 2 months, at least 3 months, at least 4 months, at least 5 months, at least 6 months, at least 12 months, at least one year, or for more than one year.

Embodiment 37. An ophthalmic composition comprising (a) lifitegrast and (b) at least one semifluorinated alkane, for use in a method for the treatment or temporary prevention of at least one ocular disorder (for example, according to any one of Embodiments 25-36).

Embodiment 38. The use of (a) lifitegrast and (b) at least one semifluorinated alkane in the manufacture of an ophthalmic composition for the treatment or temporary prevention of at least one ocular disorder (for example, according to any one of Embodiments 25-36).

### Exemplary Container Closure Systems and Kits:

Embodiment 39. A container closure system containing an ophthalmic composition comprising (a) lifitegrast and (b) at least one semifluorinated alkane.

Embodiment 40. The container closure system of Embodiment 39, wherein the ophthalmic composition is the ophthalmic composition according to any of Embodiments 1-24.

Embodiment 41. The container closure system according to any one of Embodiments 39-40, wherein the container closure system is chosen from vials, ampoules, bottles, tubes, syringes, and dispenser packages.

Embodiment 42. The container closure system according to any one of Embodiments 39-41, wherein the container closure system is a polypropylene (PP) container closure system.

Embodiment 43. A kit comprising: (i) a formulation container closure system containing an ophthalmic composition comprising (a) lifitegrast and (b) at least one semifluorinated alkane and (ii) a formulation container closure system containing a delivery vehicle for dilution or suspension of the ophthalmic composition in the formulation container.

Embodiment 44. The kit of Embodiment 43, wherein the ophthalmic composition is the ophthalmic composition according to any of Embodiments 1-24.

Embodiment 45. The kit according to any one of Embodiments 43-44, wherein the formulation container closure system is chosen from vials, ampoules, bottles, tubes, syringes, and dispenser packages.

Embodiment 46. The kit according to any one of Embodiments 43-45, wherein the formulation container closure system is a PP container closure system.

It will be understood that the inventors' work has been described by way of example only and modifications may be made whilst remaining within the scope and spirit of the invention.

1. An ophthalmic composition comprising: (a) lifitegrast and (b) at least one semifluorinated alkane.

2. The ophthalmic composition of claim 1, consisting essentially of: (a) lifitegrast and (b) at least one semifluorinated alkane.

3. The ophthalmic composition of claim 2, consisting essentially of: (a) lifitegrast and (b) no more than two semifluorinated alkanes.

4. The ophthalmic composition according to claim 1, wherein the at least one semifluorinated alkane is chosen from F4H4, F4H5, F4H6, F4H7, F4H8, F5H4, F5H5, F5H6, F5H7, F5H8, F6H2, F6H4, F6H6, F6H7, F6H8, F6H9, F6H10, F6H12, F8H8, F8H10, F8H12, F10H10, or any combination thereof.

5. The ophthalmic composition according to claim 1, wherein the at least one semifluorinated alkane is 1-perfluorohexyl-octane (F6H8).

6. The ophthalmic composition according to claim 1, wherein the lifitegrast is included in the composition at a concentration from about 0.005% (w/w) to about 5% (w/w).

7. The ophthalmic composition according to claim 1, wherein the composition further comprises at least one non-therapeutic component and/or pharmaceutically acceptable excipient chosen from: tonicity agents, preservatives, buffers, pH adjustors, anti-oxidants, delivery vehicles, stabilizers, suspending agents, viscosity-increasing agents, wetting agents, solubilizing agents, chelating agents, nitrous oxide inhibitors, isotonic agents, humectants, surfactants, or any combination thereof.

8. The ophthalmic composition according to claim 1, wherein the composition is in the form of a suspension.

9. The ophthalmic composition according to claim 1, wherein the composition is substantially free of an anti-oxidant and/or substantially free of a preservative.

10. The ophthalmic composition according to claim 1, wherein the composition is non-aqueous.

11. The ophthalmic composition according to claim 1, consisting essentially of: (a) lifitegrast and (b) perfluorohexyloctane.

12. The ophthalmic composition according to claim 11, wherein lifitegrast is present at 2-5% (w/w).

13. A method for the treatment or temporary prevention of at least one ocular disorder comprising administering to a patient in need thereof an effective amount of an ophthalmic composition comprising (a) lifitegrast and (b) at least one semifluorinated alkane.

14. The method according to claim 15, wherein the at least one semifluorinated alkane in the ophthalmic composition is chosen from F4H5, F4H6, F5H6, F5H7, F6H6, F6H7, F6H8, or any combination thereof.

15. The method according to claim 13, wherein the at least one ocular disorder is dry eye disease.

16. (canceled)

17. The method according to claim 15, wherein the ophthalmic composition is topically administered to the ocular surface of the patient, or topically administered to the cornea of the patient, or instilled into the conjunctival sac of the patient.

18. The method according to claim 15, wherein the ophthalmic composition is administered in the form eye drops.

19. The method according to claim 18, wherein the ophthalmic composition is administered as a single droplet per eye, the droplet having a volume of 8-15 μL.

20. The method according to claim 15, wherein the ophthalmic composition is administered to the patient in a single dose per day, in two doses per day, in three doses per day, in four doses per day, or in up to ten doses per day.

21. A container closure system containing an ophthalmic composition comprising (a) lifitegrast and (b) at least one semifluorinated alkane.

22. The container closure system according to claim 21, wherein the container closure system is a bottle with a droplet-forming outlet.

23. The container closure system of claim 22, wherein the bottle contains at least 1 mL of the ophthalmic composition, and wherein the ophthalmic composition is preservative-free.

24-30. (canceled)
